(19) United States
(12) Patent Application Publication
(10) Pub. No.: US 2025/0325325 A1
(43) Pub. Date: Oct. 23, 2025

(54) MULTI-MODAL OCT-NIRS CATHETER FOR TISSUE ASSESSMENT AND ABLATION

(71) Applicants: CASE WESTERN RESERVE UNIVERSITY, Cleveland, OH (US); The Trustees of Columbia University in the City of New York, New York, NY (US)

(72) Inventors: Andrew M. Rollins, Highland Heights, OH (US); Michael Douglass, Cleveland Heights, OH (US); Christine P. Hendon, Bronx, NY (US); Reza Mohammadpour, Willoughby Hills, OH (US)

(21) Appl. No.: 19/186,123

(22) Filed: Apr. 22, 2025

Related U.S. Application Data
(60) Provisional application No. 63/637,042, filed on Apr. 22, 2024.
(60) Provisional application No. 63/639,806, filed on Apr. 29, 2024.

(51) Int. Cl. A61B 18/14; A61B 5/00; A61B 17/00; A61B 18/00

(52) U.S. Cl. CPC A61B 18/1492; A61B 5/0066; A61B 5/0075; A61B 2017/00061; A61B 2018/00351; A61B 2018/00577; A61B 2018/00613; A61B 2018/00648; A61B 2018/00982; A61B 2218/002

(57) ABSTRACT

In a described example, an ablation catheter can include an elongate tubular body having a distal tip portion, defining an ablation electrode. A shaft extends from the distal tip portion and a central lumen extends through the shaft. The ablation electrode includes an arrangement of apertures extending through the distal tip portion radially outwardly from the central opening. An optical coherence tomography (OCT) imaging probe extends within the central lumen and terminates in an optical assembly at a distal end thereof. A near-infrared spectroscopy (NIRS) apparatus includes a plurality of optical fibers, in which each of the optical fibers extends longitudinally through the elongate tubular body spaced radially outwardly from the OCT imaging probe and terminates in a respective distal end within at least one of the apertures in the distal tip portion.

### CROSS-REFERENCE TO RELATED APPLICATIONS

This application claims the benefit of priority to U.S. Provisional Application No. 63/637,042, filed Apr. 22, 2024, and to U.S. Provisional Application No. 63/639,806, filed Apr. 29, 2024, each of which applications is incorporated herein by reference in its entirety.

### GOVERNMENT FUNDING

This invention was made with government support under HL 149369 awarded by the National Institutes of Health. The government has certain rights in the invention.

### TECHNICAL FIELD

This application relates to a multi-modal catheter device, systems, and methods to assess tissue and/or perform ablation.

### BACKGROUND

Atrial fibrillation (AF) is a common sustained arrhythmia throughout much of the world. A common surgical treatment choice using radiofrequency (RF) or other types of ablation has become a common procedure to treat AF as well as other forms of cardiac arrhythmias and diseases. The efficacy of the ablation procedure relies on transmurality of individual lesions. However, current lesion formation typically is guided only with indirect information (e.g. temperature, impedance, contact force, etc.), which may lead to non-transmural or incomplete lesions. As a result, there can be an increased recurrence of the underlying arrhythmia.

### SUMMARY

This application relates to a multi-modal catheter device, systems, and methods to assess tissue and/or perform ablation.

An ablation catheter includes an elongate tubular body having a distal tip portion, defining an ablation electrode, and a shaft extending proximally from the distal tip portion to a proximal end of the shaft. A central lumen extends through the shaft and the distal tip portion to provide a central opening at a distal end of the distal tip portion. The ablation electrode has a substantially cylindrical body circumscribing the central lumen and extending axially from a distal end of the shaft to terminate at the distal end of the distal tip portion. The ablation electrode can also include an arrangement of apertures extending through the distal tip portion radially outwardly from the central opening. An optical coherence tomography (OCT) imaging probe extends within the central lumen of the elongate tubular body and terminating in an optical assembly at a distal end thereof that is at or spaced proximally from the distal end of the distal tip portion. A near-infrared spectroscopy (NIRS) apparatus includes a plurality of optical fibers. Each of the optical fibers extends longitudinally through the elongate tubular body spaced from the OCT imaging probe and terminates in a respective distal end within at least one of the apertures in the distal tip portion.

In another example, a method includes positioning a distal tip portion of a catheter proximal (e.g., near or in contact with) a region of interest of biological tissue. The catheter includes an elongate tubular body that terminates at the distal tip portion, at least a portion of the distal tip portion defines an ablation electrode of the catheter. The distal tip portion includes an optical coherence tomography (OCT) imaging probe extending through a central lumen of the tubular body and terminating in an optical assembly at a distal end thereof that is at or spaced proximally from the distal end of the distal tip portion. The distal tip portion also includes a near-infrared spectroscopy (NIRS) apparatus comprising a plurality of optical fibers extending longitudinally through the elongate tubular body spaced from the OCT imaging probe and terminating in a respective distal end thereof within the distal tip portion. The method can also include performing OCT imaging with the OCT imaging probe to provide OCT image data representative of one or more optical properties of the biological tissue within a field of view of the OCT probe. The method can also include performing NIRS with the NIRS apparatus to provide NIRS data representative of one or more optical properties of the biological tissue within a field of view of the NIRS apparatus. The method can also include controlling delivery of ablation energy to the ablation electrode based on at least one of the OCT image data and the NIRS data.

### BRIEF DESCRIPTION OF DRAWINGS

FIG. 1 is an assembly view showing parts of an example multi-modal catheter.

FIG. 2 is a perspective view of a distal tip portion of the catheter of FIG. 1.

FIG. 3 is a front view of an outer portion of the distal tip portion of FIG. 2.

FIG. 4 is a perspective assembly view of the distal tip portion of the catheter.

FIG. 5 is a front view of an insert portion of the distal tip portion of the catheter.

FIG. 6 is a back view of the insert portion of FIG. 5.

FIG. 7 is a front end view of the catheter of FIG. 1 with a portion of the tip removed.

FIG. 8 is a side perspective and partially exploded view of the catheter of FIG. 7.

FIG. 9 is side perspective sectional view of a distal portion of the catheter of FIG. 1 taken along line 9-9.

FIG. 10 is a side sectional view of the distal tip portion of the catheter of FIG. 2 taken along line 10-10.

FIG. 11 is a block diagram of a catheter system depicting an example optical subsystem.

FIG. 12 depicts examples of graphs depicting metrics that can be generated by a multi-modal catheter system.

FIG. 13 is a block diagram of a multi-modal catheter system.

### DETAILED DESCRIPTION

This description relates to a multi-modal catheter device, systems, and methods to assess tissue and/or perform ablation.

As an example, the multi-modal catheter device includes an optical coherence tomography (OCT) probe, near infrared spectroscopy (NIRS) apparatus, and one or more ablation electrodes. The OCT probe is configured to acquire OCT images of a sample (e.g., biological tissue, such as heart, lung, breast, thyroid, liver or other parts of the body). The OCT probe can be configured to operate as a polarization sensitive OCT probe. Also, or alternatively, the OCT probe can be configured to operate as a standard OCT probe, measuring conventional OCT scattering intensity of the sample. The acquired OCT images of the sample can be analyzed (e.g., by a computer) to assess tissue and/or optical properties (e.g., birefringence, retardance, and optical axis angle). The NIRS apparatus is configured to perform NIRS for a field of view that can overlap at least partially (or fully) with the field of view of the OCT probe. The NIRS apparatus is configured to provide NIRS data representative of a measure of the sample's absorption and scattering for wavelengths in the visible and near infrared. The one or more ablation electrodes are configured to deliver ablation energy to the sample. In some examples, the delivery of ablation energy to the sample (e.g., tissue, such a cardiac tissue) can be monitored by the OCT probe and the NIRS apparatus, which can provide real-time feedback to control the ablation energy. In addition to the OCT probe and NIRS apparatus, and ablation electrode(s), the catheter can further include one or more other ablation catheter sensing components, which may be internal or external to the catheter body. Examples of some other ablation catheter components include temperature sensors, acoustic sensors, force or contact sensors, electrodes (e.g., for sensing electrophysiology signals, sensing impedance, and/or position sensing), and irrigation. The catheter can also interface with an ablation unit, which can be a commercially available or proprietary ablation unit configured to process signals sensed by one or more ablation features.

As a further example, an ablation catheter device can include an elongate tubular body having a distal tip portion, which defines an ablation electrode. The catheter also includes a shaft extending proximally from the distal tip portion to a proximal end which can attach to a handle. A central lumen can extend through the shaft and the distal tip portion to provide a central opening at a distal end of the distal tip portion. The ablation electrode can have a substantially cylindrical body (or other shape) circumscribing the central lumen. The ablation electrode further can include an arrangement of apertures extending through the distal tip portion spaced radially outwardly from (e.g., surrounding) the central opening. The ablation catheter can also include an OCT imaging probe extending through the central lumen of the elongate tubular body and terminating in an optical assembly at or spaced axially proximally from the distal end of the distal tip portion. The OCT imaging probe can be configured to perform PSOCT, spectrometric OCT, dynamic OCT, and/or Doppler OCT (DOCT) based on optical signals transmitted to and from an associated OCT apparatus. The NIRS apparatus includes a plurality of optical fibers (e.g., at least one fiber for illumination and at least one fiber for detecting spectroscopic information). Each of the optical fibers can extend longitudinally through the elongate tubular body of the catheter, such as spaced radially outwardly from the OCT imaging probe, and terminate in a respective distal end thereof that can be within at least one of the apertures in the distal tip portion. Each of the OCT imaging probe and the fibers of the NIRS apparatus can be coupled with an optical subsystem (or multiple subsystems) to convert the optical signals into respective OCT data and NIRS data. A controller can be configured to control delivery of energy to the ablation electrode based on the OCT data and/or the NIRS data. Also, or as an alternative, a computer or other processor-based device can be configured to generate one or more visualizations based on the OCT data and/or the NIRS data, which can be prior to, during, and/or after delivery of energy to the ablation electrode. In examples herein, the catheter can be further compatible with 3D electroanatomical mapping systems. Therefore, optical and/or tissue properties measured by the probe can be mapped onto the 3D map generated by these systems, so that 3D maps of optical and/or tissue properties are generated and available to the clinician (e.g., superimposed on a graphical representation of anatomy, an electroanatomical map, and tissue properties derived from NIRS and/or OCT data).

As described herein, by integrating OCT (e.g., PSOCT) and NIRS modalities into the multi-modal catheter, imaging depth and/or analysis of lesion quality can be improved compared to existing approaches. Also, the multi-modal catheter can be used to monitor OCT/PSOCT, NIRS, and other ablation feedback parameters concurrently for a common region of interest (e.g., a respective lesion). The multi-modal catheter, systems, and methods can improve the treatment of cardiac arrhythmias (e.g., atrial fibrillation, atrial flutter, ventricular tachycardia, ventricular fibrillation, etc.) by monitoring properties of tissue and/or lesion quality (e.g., transmurality and/or diameter) prior to, during (e.g., by providing intraoperative feedback), and/or after the delivery of ablation to a tissue substrate. The systems and methods herein further can facilitate searching and identifying pathological regions for therapeutic treatment.

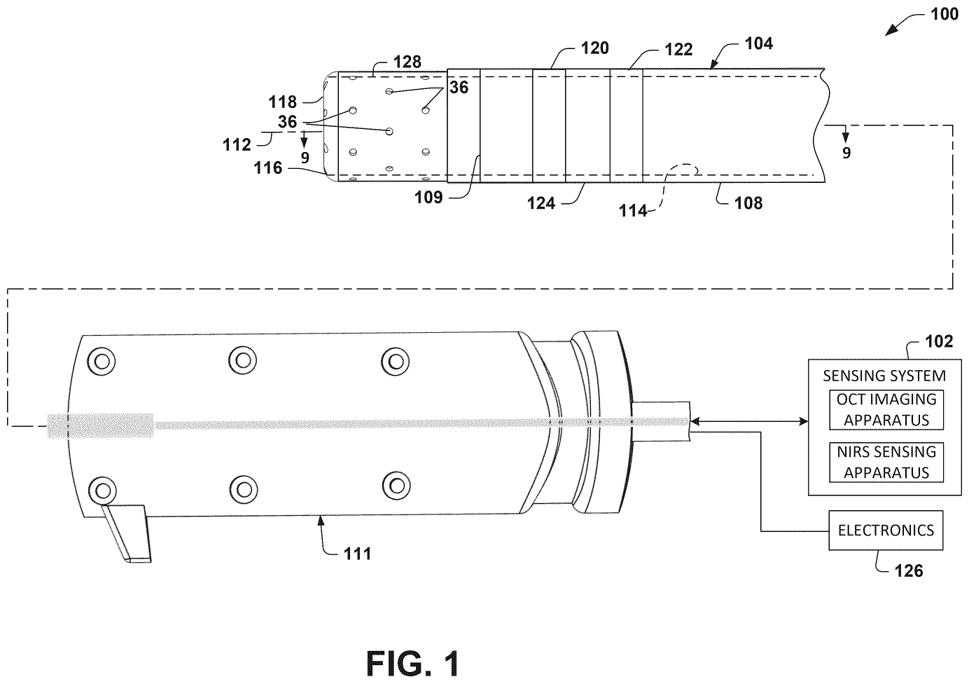

An example multi-modal ablation catheter will be better appreciated with reference to FIGS. 1-10, which illustrate various views of an example catheter. Turning to FIG. 1, a multi-modal catheter 100 is shown coupled to a sensing system 102 and associated electronics 126. The multi-modal catheter 100 includes an elongate tubular body 104 having a distal tip portion 106 and a shaft 108. A distal end 109 of the shaft 108 can be coupled to a proximal end 110 of the distal tip portion 106 (e.g., by an adhesive or other coupling, such as heat bond, weld, etc.). The shaft 108 can extend proximally from the distal tip portion 106 to a proximal end of the shaft that is coupled to a handle 111. A central axis 112 can extend longitudinally through the tubular body 104, including through the shaft 108 and the distal tip portion 106.

The handle 111 can provide a portion that can be used to grasp and adjust the position of the distal tip portion 106 or another portion of the shaft 108 of the catheter 100. The handle 111 can also be implemented as a mechanical control handle having one or more knobs (or other control mechanisms) configured to adjust the length of respective pull wires for steering the distal end portion of the catheter 100. For example, rotating the knob in one direction can cause deflection of the distal end portion in a first direction transverse to the axis 112 and rotating the knob in another direction causes deflection in another (e.g., opposite) direction.

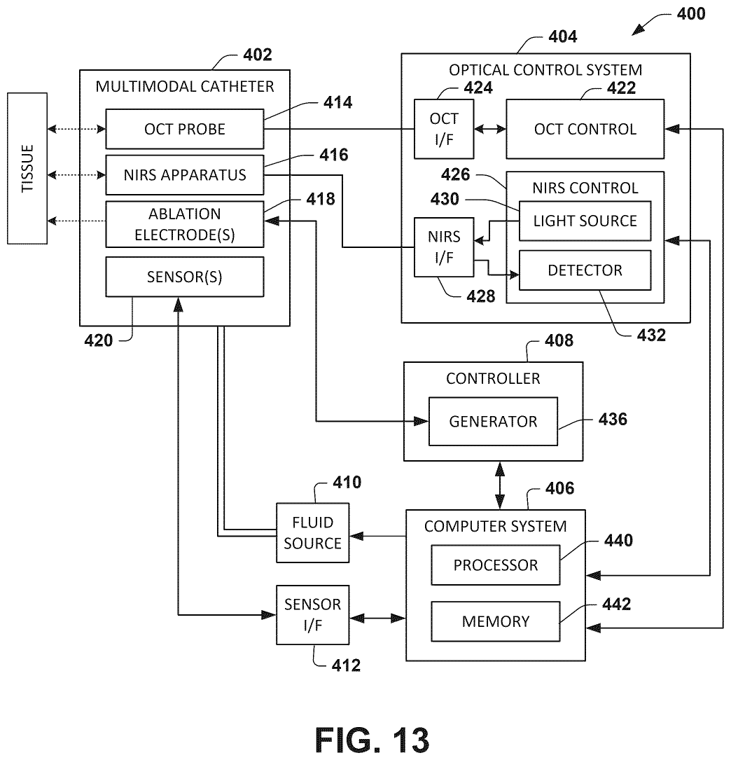

The distal tip portion 106 can itself define one or more ablation electrodes, which are adapted to implement one or more types of ablation based on ablation energy received from a generator (e.g., can be part of electronics 126—see also FIG. 13). A central lumen 114 extends through the tubular body 104, including through the distal tip portion 106 and the shaft 108, to provide a central opening 116 at a distal end 118 of the distal tip portion 106. The distal end 118 of the distal tip portion 106 can have chamfered or curved edges extending from a radially outer sidewall 128 of the distal tip portion 106 to the central opening 116. Also, or alternatively, the distal end 118 can be rounded (e.g., semi-spherical).

In the example of FIG. 1, the catheter 100 can also include one or more electrodes 120 and 122 disposed on or embedded within the shaft 108. For example, the electrodes 120 and 122 can be band (or ring) electrodes that circumscribe a radially outer sidewall 124 of the shaft 108 at axially spaced apart locations. Other shapes and numbers of electrodes (e.g., one, three or more electrodes) can be used in other examples. The electrodes 120 and 122 can be formed of an electrically conductive material. The electrodes 120 and 122 can be implemented as sensing electrodes, which can be coupled to sensing and/or other electronics 126 through one or more wires or conductive traces that extend axially through the tubular body 104 and handle 111. Also, or as an alternative, one or more of the electrodes 120 and 122 can be implemented as ablation electrodes, which can be electrically coupled with or isolated from the ablation electrode of the distal tip portion 106. The relative position of the distal end 118 (or other parts of the catheter 100) and each of the electrodes 120 and 122 can be known, such as to enable localization of the distal end 118 and/or other parts of the catheter 100 (e.g., by a localization or navigation system that could be part of the electronics 126).

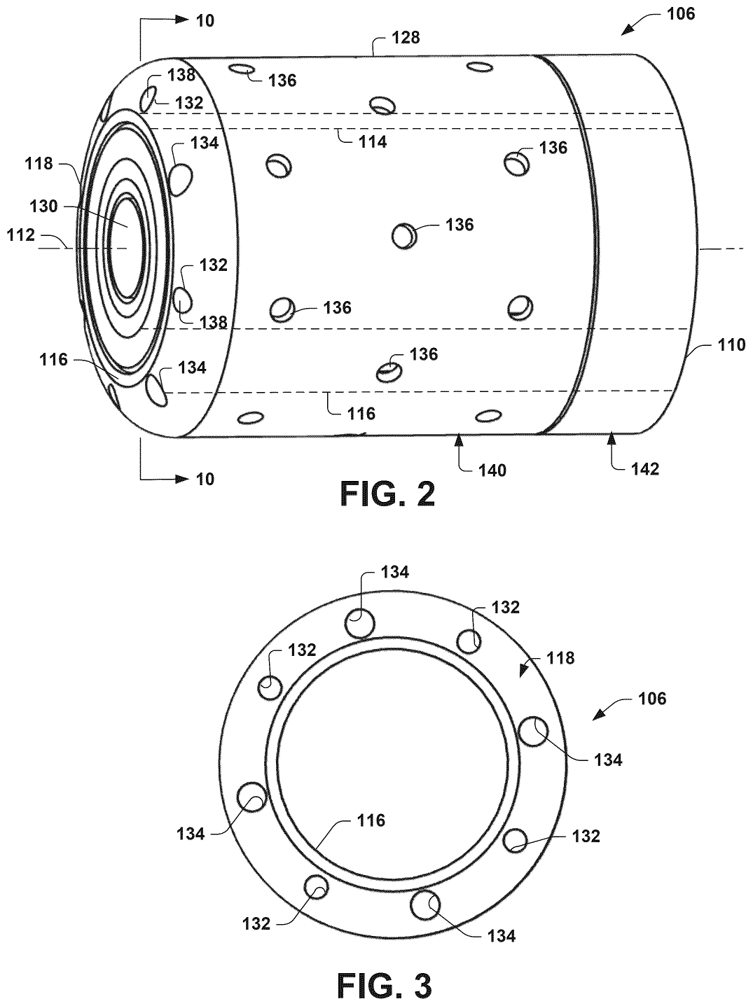

FIG. 2 illustrates a side perspective view of the distal tip portion 106 showing the distal end 118 (e.g., front end) of the catheter 100. As described herein, the catheter 100 includes an OCT imaging probe and a NIRS apparatus integrated into the elongate tubular body 104 of the catheter. In FIG. 2 a window 130 of an optically transparent material is mounted within the central opening 116 at the distal end 118. The window 130 can form a protective cover for (or part of) an optical assembly of the OCT imaging probe. The window 130 has a front face at the distal end 118 and a back face (not shown—but see FIGS. 8-10) within the lumen 114 spaced axially proximally from the opening 116 at the distal end. In an example, the window glass can be fixed to the distal tip portion with optical adhesive (e.g., at glue joints separated by an angle of about 120° around the window). Other means for fixing the window with respect to opening 116 (e.g., friction fitting, couplings, brackets, fasteners, etc.) can be used in other examples.

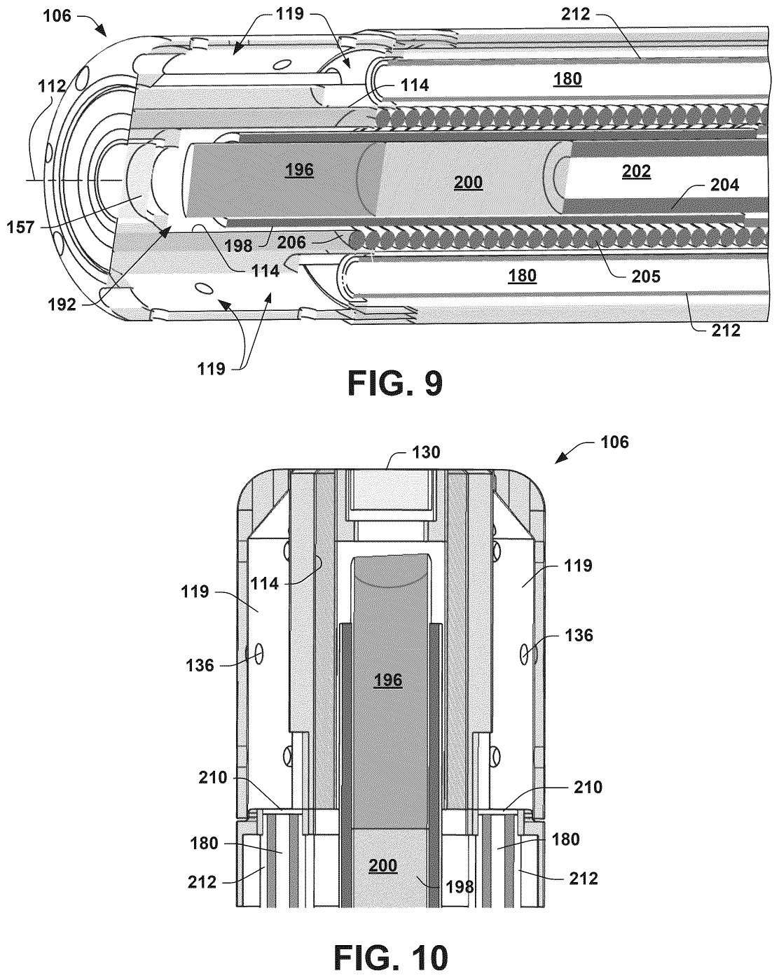

As shown in FIGS. 1 and 2, the distal tip portion 106 includes a substantially cylindrical sidewall 128 extending between proximal and distal ends 110 and 118 and circumscribing the central lumen 114 and extending axially from a distal end of the shaft to terminate at the distal end of the distal tip portion. The distal tip portion 106 can also include a plurality of apertures 132, 134 extending through the distal end 118 of the distal tip portion 106 arranged around (e.g., circumscribing) the central opening 116. For example, one group of the apertures 132 can define terminations for mounting respective optical fibers of a NIRS apparatus, which fibers extend through the elongated shaft 108. Another group of the apertures 134 through the distal end 118 can define irrigation ports that are fluidly connected with an interior volume (e.g., an interstitial space 119—see FIGS. 9 and 10) within the distal tip portion 106. The distal apertures 134 thus can be configured to direct irrigation fluid in a substantially axial direction. For example, the group of apertures 134 are distributed substantially evenly and circumscribe a periphery (e.g., along an inner sidewall surface) of the central opening 116 such as to hold distal ends of respective optical fibers 138 circumferentially around the central lumen 114, which contains the window 130 and optical assembly of the OCT imaging probe.

FIG. 3 illustrates a front end view of the distal tip portion 106 with the window 130 and other components (e.g., optical fibers, optical assembly removed. In the example of FIGS. 2 and 3, the apertures 132 and 134 are spaced in an alternating manner circumscribing the central lumen 114. Other numbers and arrangements of apertures can be used in other examples.

The distal tip portion 106 can include another set of apertures (also referred to as sidewall apertures) 136 that extend through the sidewall 128 at distributed locations. The sidewall apertures 136 can define a second set of irrigation ports configured to enable the flow of irrigation fluid outwardly from within the interior volume (see, e.g., volume 119 of FIGS. 9 and 10) through the sidewall 128. The sidewall apertures 136 can be substantially evenly distributed across the sidewall, such as shown, or the apertures can be arranged a prescribed pattern for directing irrigation fluid in one or more particular directions with respect to the sidewall. The size, number, and distribution of the apertures 136 along the sidewall 128 can vary depending on desired rate of fluid flow and the size of the distal tip portion 106.

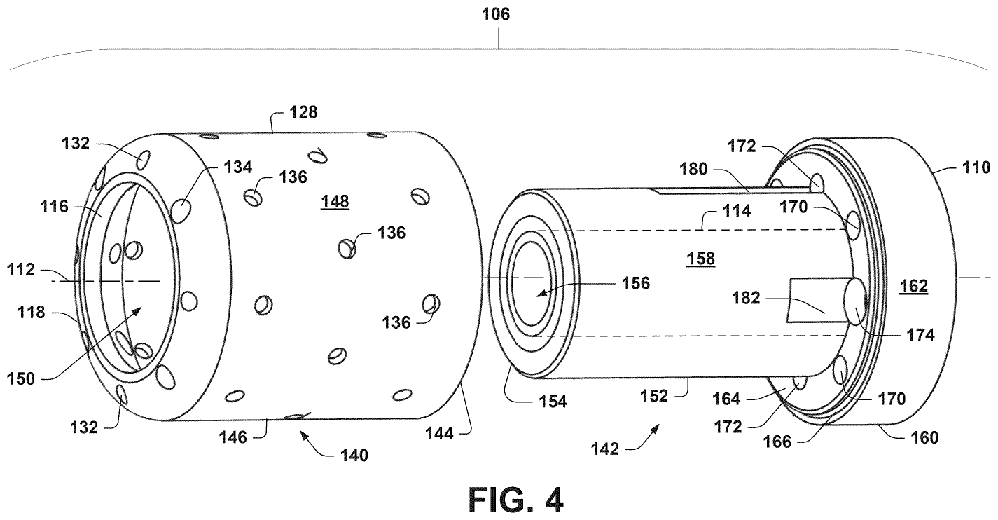

In some examples, the distal tip portion 106 can be formed of two or more primary portions. As shown in the example of FIG. 4, the distal tip portion 106 can be formed of first and second primary housing portions 140 and 142, which can be referred to, respectively, as a NIRS shell and an insert electrode. The multiple component configuration for the distal tip portion 106 can facilitate assembly and construction of the respective optical components into the multi-modal catheter 100. While the example of FIG. 4 shows two separate main tip portions 140 and 142, in other examples, the distal tip could be implemented as a single part (e.g., produced by additive manufacturing methods like 3D printing).

One or both of the respective portions 140 and 142 can be formed of electrically conductive materials, in which each electrically conductive portion can define the ablation electrode. In examples where each of first and second portions 140 and 142 are formed of electrically conductive materials, the portions can be electrically coupled to each other, such as to define a single electrode structure. In other examples where each of first and second portions 140 and 142 is formed of electrically conductive materials, the respective portions are electrically isolated from each other, such as by interposing an insulating material between the respective adjacent contacting surfaces. In examples where each of first and second portions 140 and 142 are electrically isolated from each other, the respective portions can form a bipolar pair of electrodes, such as for bipolar pulsed field ablation (PFA). In other examples, another electrode (e.g., one of the band electrodes or a body surface electrode) can be used as another electrode for bipolar PFA.

As shown in FIG. 4, the first portion 140 has a proximal end 144 and distal end, which corresponds to the distal end 118, and a substantially cylindrical first sidewall 146 extending axially between the proximal and distal ends. The first sidewall 146 has an outer periphery 148 and an inner periphery 150 spaced radially inwardly from the outer periphery. The inner and outer peripheries 150 and 148 can be substantially concentric cylinders. The first portion includes, as its distal end, the distal end 118 through which the apertures 132 and 134 and the central opening 116 extend.

As shown in FIG. 4, the second portion 142 has a substantially cylindrical second sidewall 152 that extends between a proximal end, which corresponds to the proximal end 110, and a distal end 154. The second sidewall 152 has an inner periphery 156 and an outer periphery 158. The central lumen 114 of the distal tip portion 106 extends axially through a central portion of the second sidewall and defines at least a portion of the inner periphery 156 of the second sidewall 152. In some examples, a ledge 157 can be formed along the inner periphery 156 adjacent to and spaced axially a distance apart from the distal end 154 to receive and hold the window 130 at a desired location. In the example of FIG. 4, the second portion 142 is configured to be inserted axially into an interior of the first portion 140 to form the assembled distal tip portion 106, as shown in FIG. 2. Thus, when assembled, at least a portion of the outer periphery 158 of the second sidewall 152 is spaced radially inwardly from and coextensive with the inner periphery 150 of the first sidewall 128 and the coextensive space between the first and second portions 140 and 142 define a cylindrically shaped volume (e.g., volume 119 shown in FIGS. 9 and 10) of the distal tip portion between the first portion and the second portion. Also, the central lumen 114 defined by the inner periphery 156 of the second sidewall 152 is configured to receive therein a distal portion of the optical assembly of the OCT probe, and the proximal end 110 of the sidewall 152 of the second portion 142 is coupled to the distal end of the shaft.

In some examples, the second portion 142 includes an annular base 160 at the proximal end 110. The annular base 160 is coaxial with and has a larger diameter than the outer periphery 158 of the second sidewall 152, which extends axially distally from the annular base. The central lumen 114 also extends through the annular base 160. The annular base 160 has a sidewall portion 162 that extends radially outwardly from the outer periphery 158 of the second sidewall 152, such as to define a flange (or a ledge) along the distal end 164 thereof. The sidewall portion 162 of the annular base 160 and the sidewall 146 of the first portion 140 can have the same or approximately the same outer diameter. The sidewall portion 162 of the annular base 160 extends axially between the proximal end 110 of the second portion 142 and a distal end 164 of the annular base to define a length thereof. The axial length of the sidewall portion 162 can approximate a width of an electrode band on an outer surface of the catheter.

As mentioned, the portion of the sidewall 152 extending between the distal end 164 of the annular base and the distal end 154 of the sidewall 152 is adapted for insertion into the interior of the first portion 140 of the distal tip portion 106. The length of the outer periphery of the second sidewall 152 that extends axially from the annular base 160 can be equal to or approximate the axial length of the sidewall 146 of the first portion 140, such that when assembled, the distal end 154 is substantially flush with the distal end 118 of the first portion (see, e.g., FIG. 2). A radially outer distal edge 166 of the annular base 160 can be configured to connect to a proximal edge of the sidewall 146. The annular base 160 can define a mating end cap at the proximal end of the distal tip portion 106 to attach the first and second portions together, and laser welding (or other fastening means, such as adhesives, heat bonding, etc.) can fix the attachment. The sidewall 146 of the first portion 140 and the outer surface of the sidewall portion 162 of the annular base 160 can form the sidewall 128 of the distal tip portion 106 when assembled together, such as shown in FIGS. 1 and 2.

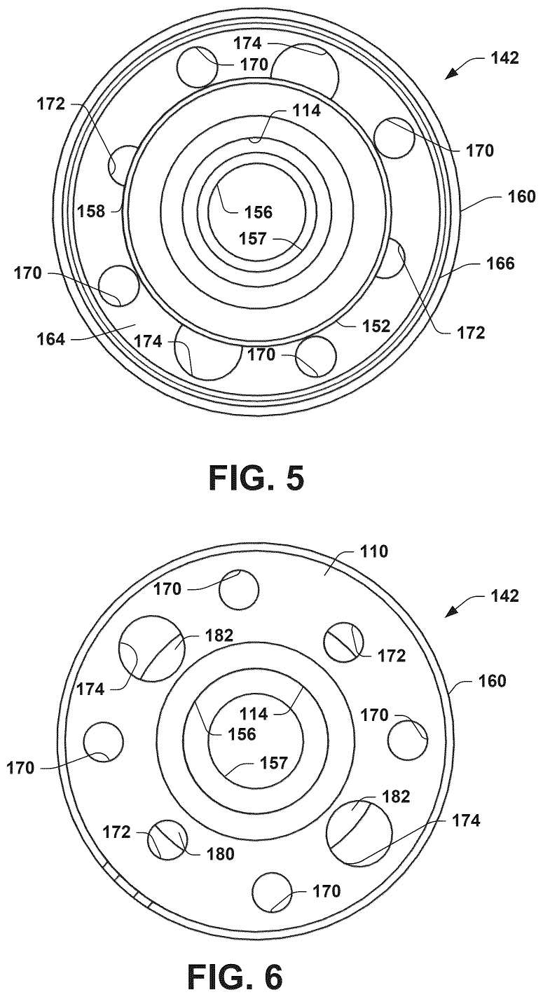

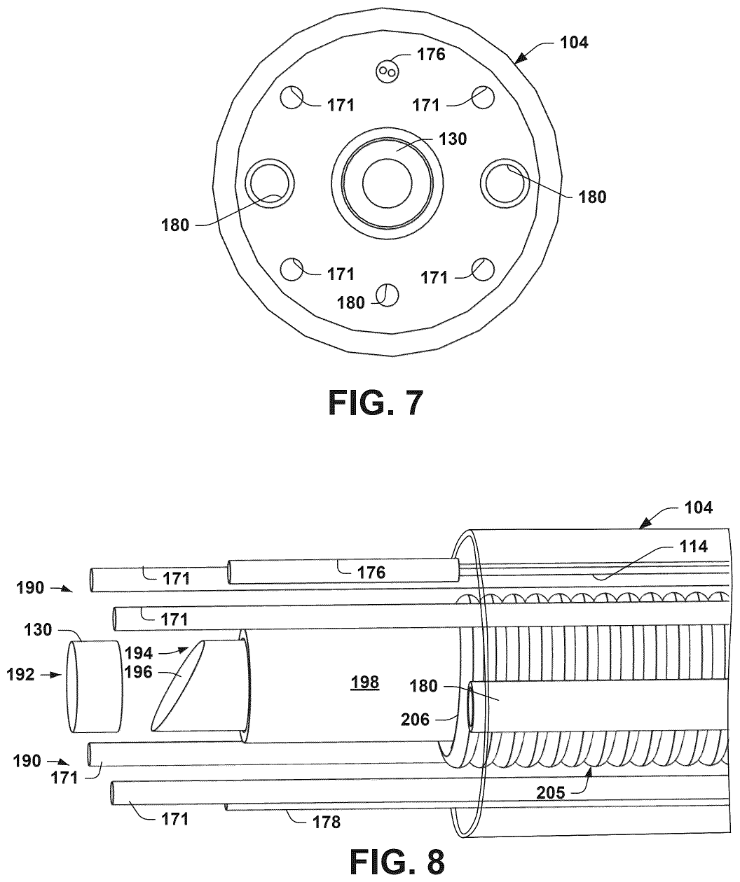

FIG. 5 is front end view of the second portion 142 (e.g., looking at the distal end 154) and FIG. 6 is back end view of the second portion (e.g., looking from at proximal end 110). As shown in FIGS. 4, 5, and 6, the annular base 160 can also include an arrangement of apertures 170, 172, and 174 (also referred to as slots) extending axially through the of the annular base circumscribing the inner periphery 156 for holding various components that extend through the elongated shaft 108 of the catheter 100. For example, one group of the apertures 170 can define guides for receiving respective optical fibers (e.g., optical fibers 171 shown in FIGS. 7 and 8) of a NIRS apparatus. As described herein, the optical fibers 171 can extend through the elongated shaft 108 of the catheter and the apertures for such fibers are configured to reduce strain such as to prevent fibers from breaking. The distal end of such optical fibers 171 can be mounted within respective apertures 132 of the first portion 140 of the distal tip portion 106. Thus, when assembled, the apertures 170 axially align with the apertures 132.

Other groups of the apertures 172 and 174 are configured to receive components therein which can be held in place with respect to the second portion 142. For example, the apertures 172 can be configured to receive an RF electrode 178 and a thermocouple (or other temperature sensor) 176, such as. While two such apertures 172 are shown in the example of FIGS. 5 and 6, other numbers of apertures can be used for other components/sensors. The other, larger diameter apertures 174 each can receive therein an irrigation tube 180 that carries irrigation (e.g., cooling) fluid from a fluid source (e.g., a controllable source of cooling fluid—see FIG. 13). As shown in the example of FIGS. 4 and 6, slots (or channels) 182 and 184 can be formed in the outer periphery 158 of the second sidewall 152 extending from and coaxially aligned with respective apertures (at the distal end 164). The slots 182 and 184 can have the same diameter as the apertures 172 and 172, respectively, from which the slots extend. The slots 182 and 184 can define joints to which an adhesive (or other fastening means) can be applied to fix the respective components (e.g., RF electrode 178, thermocouple 176, irrigation tubing 180) along the outer periphery 158 of the second sidewall 152.

The apertures 170, 172, and 174 and slots 182 and 184 can be formed by machining (e.g., drilling or laser cutting) through the annular base 160 at desired locations. Further, guides can be provided along the outer periphery 158 of the second sidewall 152 for optical fibers to provide strain relief. Also, or as an alternative, an interior of the second portion can include features (e.g., guides along the inner periphery 156) to center the OCT probe, including the optical assembly thereof, as well as to maintain a fixed axial distance between a back face of the window 130 and the distal end of the OCT probe (the lens thereof). The interior of the second portion 142 further can be configured (e.g., ledges and the like) to provide a bearing surface during rotation of the OCT probe therein. By constructing the first and second portions 140 and 142 (NIRS shell and insert electrode), respectively, as separate components of the distal tip portion 106, the optical fibers 171 for the NIRS apparatus can be polished without fouling or otherwise adversely impacting other optical components (e.g., the OCT probe) or joints in the second portion 142.

An example configuration for a NIRS apparatus 190 and OCT probe 192 as well as other interior features of the catheter 100 will be better appreciated with respect to FIGS. 7, 8, 9, and 10. FIG. 7 is a front end view of the catheter with the distal tip portion 106 removed. FIG. 8 is a side view of FIG. 7 showing a distal portion of the catheter (also without the distal tip portion). FIG. 9 is side perspective sectional view of a distal portion of the catheter of FIG. 1 taken along line 9-9 showing the arrangement of features in the distal portion of the catheter. FIG. 10 is a side sectional view of the distal tip portion 106 of the catheter of FIG. 2 taken along line 10-10, showing the arrangement of features therein.

Referring to FIGS. 7 and 8, OCT probe 192 extends longitudinally within the central lumen 114 of the elongate tubular body 104 and terminates in an optical subassembly 194 at a distal end thereof. The optical subassembly 194 can include a lens (e.g., a grin lens) 196, which is mounted within and extends axially from a distal end of tubular sheath 198. An optical spacer 200 (e.g., a coreless optical spacer, such as including an optical pigtail) can be coupled (e.g., by an optical adhesive or other coupling) between the lens 196 and an optical fiber 202 of the OCT probe 192. The optical fiber 202 can be mounted within and extend outwardly from a torque coil 204, such as shown in FIG. 9. For example, as the OCT probe 192 rotates and is pushed forward to contact tissue, the torque coil 204 can help reduce tensile stress placed on the optical fiber. The optical fiber 202 of the OCT probe 192 can extend proximally from the pigtail within a central lumen of the torque coil 204 through the body 104 of the catheter 100 and be coupled to an interface of a sensing system 102 (e.g., OCT control apparatus 422 of FIG. 13). The tubular sheath 198 thus can contain the torque coil 204, the optical fiber 202, the spacer 200 and a portion of the lens 196. In some examples, a length of a helical coil 205 (e.g., stainless steel or other suitable material) can be mounted around the tubular sheath within the central lumen 114, and a distal portion of the sheath 198 and lens 196 can extend axially distally from the distal end 206 of the coil 205.

In some examples, the tubular sheath 198 of the optical subassembly can be formed of a pliant steel tube and is configured to center the spacer 200 to the lens 196. The lens 196 can have both faces polished (e.g., at about an 8° or other angle), minimizing back reflections and ensuring that any orientation of the GRIN lens aligns with the angle of the coreless spacer 19. The distal face of the lens 196 can be spaced proximally from the distal end 118 of the distal tip portion 106, such as at a fixed axial distance from a proximal face of the window 130, when the catheter 100 is assembled, such as shown in FIGS. 9 and 10. As described herein, the OCT probe 192, including optical subassembly 194, is configured to rotate about the central axis 112 within the central lumen 114, such as for performing OCT (e.g., PSOCT image scanning. For example, the OCT probe 192 can be configured to transmit light off-axis through the window 130 and opening 116, which can be provided in a substantially conical or cylindrical beam pattern that defines a field of view of the OCT probe. The transmitted light can be provided with a spot size on a sample, which can be tuned to a desired size along with the size and shape of the beam pattern. The NIRS apparatus 190 includes the optical fibers 171, in which each of the optical fibers extends longitudinally through the elongate tubular body 104 spaced radially outwardly from and circumscribing the OCT probe 192. As described herein, the optical fibers 171 can extend through respective lumen in the shaft 108, through apertures 170 of the annular base 160 to terminate within respective apertures 132 of the first portion 140 of the distal tip portion 106. The optical fibers 171 further can extend proximally though the tubular body 104 of the catheter 100 and be coupled to an interface of an optical sensing system 102 (e.g., an NIRS apparatus of FIG. 13).

In the example shown in FIGS. 1-10, the NIRS apparatus 190 is demonstrated as a forward facing device with the distal end faces of the optical fibers 171 facing axially distally. Also, or alternatively, the NIRS apparatus can be configured to be side viewing, such as having apertures that extend radially through the sidewall 128 (e.g., similar to apertures 136) near the distal end 118 of the distal tip portion 106 at locations where NIRS detection is desired. In the lateral or side-facing example, the NIRS fibers can be bent or include a total-internal reflectors to transmit and/or receive light by through respective apertures formed through the sidewall of the distal tip portion. Each NIRS fiber can have its own aperture or multiple NIRS fibers can be provided in one or more apertures (e.g., slots).

As further example, the ablation catheter 100 is configured to provide irrigation fluid to cool the distal tip portion 106 and/or nearby tissue. As described herein, one or more elongated tubes 180 extend longitudinally through the shaft 108 and terminate at distal ends 210 that are in fluid communication with the volume 119 of the distal tip portion 106. An arrangement of apertures 134 and 136 are formed through the distal end 118 and sidewall 128 of the first portion (NIRS shell) 140 to enable flow of fluid from the volume 119 to a region outside of the distal tip portion of the catheter 100. The irrigation tubing 180 can reside in one or more respective lumens 212 extending longitudinally through the shaft 108 and in fluid communication with the interior volume 119. The tubing can extend from the proximal end of the catheter shaft and be in fluid communication with one or more sources of fluid (e.g., fluid source of FIG. 13). As described herein, an irrigation controller (e.g., computer of FIG. 13) can be configured to control flow of fluid into the tubing 180 and volume 119 based on one or more measured parameters, such as sensed temperature (e.g., by temperature sensor) 176, OCT image data, and/or NIRS data.

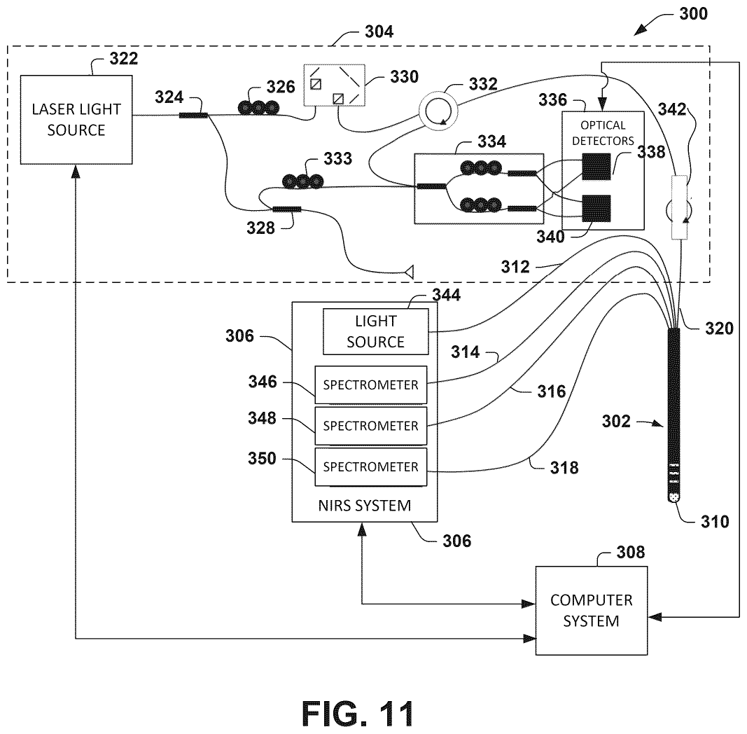

FIG. 11 depicts an example system 300 including a multi-modal catheter 302, an OCT system 304, a NIRS system 306, and a computer system 308. The computer system 308 can be in communication (directly or indirectly) with each of the catheter 302, the PSOCT system 304, the NIRS system 306, such as to monitor data being acquired and/or control functions thereof.

The catheter 302 can be implemented according to the example catheter described with respect to FIGS. 1-10. Accordingly, the description of the catheter 302 can refer to certain aspects of FIGS. 1-10. It is to be appreciated, however, that the catheter 302 can be implemented according to other configurations that differ from that described with respect to FIGS. 1-10. For example, the catheter 302 includes an OCT probe and an arrangement of NIRS fibers. The catheter 302 can also include one or more ablation electrodes, such as at a distal tip portion 310 (e.g., distal tip portion 106). In some examples, the catheter 302 can also include one or more non-optical sensors, such as band electrodes or other sensors on the surface or within the catheter body. As described herein, such one or more other sensors can be used to sense parameters and provide respective metrics (e.g., temperature, impedance, power, contact force, spatial position, or the like) for the catheter, a sample (e.g., biological tissue) and/or interaction between the catheter and the sample.

The system includes a plurality of NIRS optical fibers 312, 314, 316, and 318 and an optical probe 320 that includes one or more optical fibers. The NIRS optical fibers 312, 314, 316, and 318 are coupled to the NIRS system 306 for communicating NIRS optical signals. The OCT probe 320 and the optical fiber(s) thereof are coupled to the OCT system 304 for communicating OCT optical signals. As described herein, the NIRS fibers 312, 314, 316, and 318 and the PSOCT probe 320 are integrated into the distal tip portion 310 of the catheter 302.

In the example of FIG. 11, the OCT system 304 is described as a PSOCT interferometer having one example architecture. In other examples, different PSOCT architectures can be used. Also, or alternatively, other types of engines can be used to drive the OCT probe 320 for performing other measurement functions.

The example PSOCT interferometer includes a laser light source 322. The laser light source 322 can be a swept source laser configured to be sampled as a function of wavenumber, k. The laser light source can thus provide broadband light to a fiber coupler (e.g., splitter) 324 via a corresponding waveguide, and a portion of the broadband light can be provided to a polarization controller 326 via a corresponding waveguide, and another portion of the light to another fiber coupler (e.g., splitter) 328, which conducts the light to the reference delay line. The polarization controller 326 can be configured to adjust measured light to known polarization states for each measurement, such as to help optimize the spectrum and balance the power. The polarization controller 326 can be implemented by an automated controller or manual paddles can be used. A circulator 332 maximizes the available laser power at the tissue.

A polarization delay unit 330 is configured to separate and make the polarization states perpendicular. For example, the polarization delay unit 330 can enforce depth multiplexing of the orthogonal polarization states of the laser light. A circulator 332 can receive laser light from the polarization delay unit 330 and direct the laser light to the OCT probe 320. In some examples, the OCT system includes a rotary joint 342 for rotating the OCT probe within the catheter 302 to implement rotational scanning and provide a conical pattern beam pattern. The fiber coupler 328 can be coupled to the detection unit 334 through another polarization controller 333 to control the polarization state of the light returning from the reference delay line. The circulator 332 further can receive reflected and/or scattered light from the OCT probe 320 and conduct the light to a detection unit 334. For example, the detection unit 334 includes respective reference and input channels, from the delay line and the OCT probe, respectively, including an arrangement of polarization controllers, polarizing beam splitters that deliver light to balanced optical detectors 338 and 340, which are configured to convert light to voltage signals. The detection unit 334 is also configured to separate the light into orthogonal polarization states for further processing and analysis. The outputs from the optical detectors 338 and 340 can be provided to the computer system 308 for evaluation. As an example, the PSOCT system 304 can be configured to acquire OCT images at frame rate and resolution (e.g., about 50 frames per second or faster with about 1000 A-lines or more per frame).

The NIRS system 306 includes one or more light sources 344, and, for example, a plurality of spectrometers 346, 348, and 350. For example, the light source is a white light source for illumination. The light source 344 and spectrometers 346, 348, and 350 can each be optically coupled to a respective NIRS fiber 312, 314, 316, and 318. Alternatively, for example, a fiber optic switch could be used to multiplex the light from NIRS fiber 314, 316, and 318 to a single spectrometer. The spectrometers 346, 348, and 350 can provide NIRS measurements (e.g., NIRS data) to the computer system 308.

The computer system 308 is configured to process and analyze outputs from the optical detectors 338 and 340 and the spectrometers 346, 348, and 350. For example, the computer system 308 can be configured to determine values representing detected optical and/or other properties (e.g., birefringence and/or optic axis measurements) of the sample such as described herein. The computer system 308 can also be configured to determine NIRS properties of the sample based on the NIRS spectroscopic information, such as including a quantitative lesion optical index (LOI) or other NIRS metrics derived from measured NIRS spectra for the sample (e.g., biological tissue). The LOI is value that calculated based on a combination of spectral changes that have been determined to indicate a lesion formation (e.g., during ablation), such as described in Park S Y, Singh-Moon R, Y ang H, Saluja D, Hendon C; Quantification of irrigated lesion morphology using near-infrared spectroscopy; Sci Rep. 2021 Oct. 11; 11 (1): 20160. In some examples, the computer system 308 is configured to compute one or more indices based on a combination of the OCT image data, NIRS data, and/or other sensor data for characterizing properties or attributes of the tissue, catheter, and/or interactions between the catheter and the tissue, such as described herein.

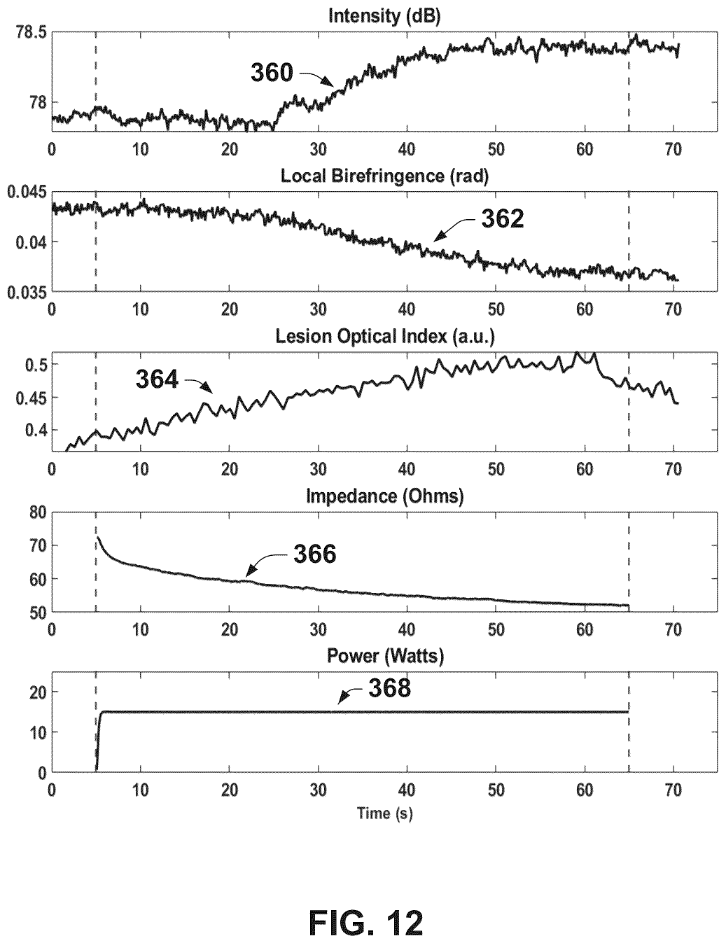

By way of example, FIG. 12 includes plots 360, 362, 364, 366, and 368 depicting example metrics that can be generated by a multi-modal catheter system (e.g., catheter system of FIGS. 1, 11, and 13) during ablation of a region target tissue based on OCT data, NIRS data and other sensor data over time. The plot 360 represents scattered light intensity over time from OCT data, which demonstrates that scattering increases during ablation. The plot 362 represents tissue birefringence over time based on PSOCT data, which demonstrates birefringence decreasing during ablation. The plot 364 represents a LOI based on NIRS data, which demonstrates NIRS spectral changes during ablation. The plot 366 shows impedance over time (based on impedance measurements from tissue), which decreased during ablation. The plot 368 shows ablation energy (power) over time, which demonstrates that power stayed substantially constant. The plots 360, 362, 364, 366, and 368 can be used to provide information based on which one or more indices can be computed (e.g., by sensing system 102, computer system 308, or computer system 406). As described herein, the indices can characterize at least one property or attribute of the sample (e.g., biological tissue), the ablation catheter, and/or an interaction between the ablation catheter and the tissue (e.g., during ablation).

FIG. 13 is a block diagram of a multi-modal catheter system 400. In the example of FIG. 13, the system 400 includes a multi-modal catheter 402, an optical control system 404, a computer system 406, an ablation controller 408. The system 400 can also include a fluid source 410 and one or more sensor interfaces 412, which can be coupled to respective components of the catheter 402.

In the example of FIG. 13, the catheter 402 includes an OCT probe 414, a NIRS apparatus 416 (e.g., including one or more optical fibers), one or more ablation electrodes 418, and one or more sensors 420. The catheter 402 (e.g., catheter 100, 302) can be implemented according to any of the examples provided herein. Accordingly, the description of FIG. 13 can also refer to certain aspects of FIGS. 1-11. Thus, by integrating at least OCT and NIRS into the distal portion of the same catheter synergistic concurrent optical measurements can be acquired of a target region of interest (e.g., biological tissue, such as cardiac tissue) in an efficient manner to facilitate targeting, delivery, and termination of ablation energy, as described herein.

The optical control system includes an OCT control apparatus 422 that includes or is optically coupled to the OCT probe 414 through an OCT interface 424. The OCT control apparatus 422 can be configured to perform OCT (e.g., OCT imaging and/or other OCT measurements) by transmitting and receiving optical signals from the OCT probe. For example, the OCT control apparatus 422 is configured to perform PSOCT imaging through the OCT probe 414 and provide OCT image data to the computing system 406 representative of one or more optical properties of across a region of a sample (e.g., biological tissue that is within a field of view (e.g., a conical image beam pattern) of the OCT probe. In an example, the OCT control apparatus 422 can be configured as described with respect to FIG. 11 for performing PSOCT.

The OCT control apparatus 422 can be configured to perform other types of OCT measurements and/or OCT imaging. As an example, the OCT control apparatus 422 can be configured to implement spectral domain OCT (SDOCT) to enable simultaneous OCT measurements in multiple wavelength windows. For example, the computer system 406 can be configured to determine information on the concentration of specific constituents of the tissue (or other sample) based on SDOCT data provided by the OCT control apparatus 422. Also, or alternatively, the OCT control apparatus 422 can be configured to implement dynamic OCT, and/or DOCT through the OCT probe 414, which can measure and/or monitor Brownian motion and flows of biologic liquids at or around the sample. For example, the computer system 406 can provide a depth resolved profile of the flow velocity in the vessel and/or a quantitative method to measure tissue perfusion based on DOCT data. Other types of OCT control apparatuses that can be used to implement the OCT control apparatus 422, including for PSOCT or other forms of OCT, are disclosed in U.S. Pat. Nos. 10,591,275, 7,826,059 and 6,615,072, each of which is incorporated herein by reference. Other OCT apparatuses can be used in other examples to implement the OCT control apparatus 422 and provide OCT data (e.g., OCT image data or other OCT measurement data) to the computing system 406.

The NIRS control apparatus 426 includes one or more light sources 430 and one or more detectors 432. The light source 430 is optically coupled to at least one of the optical fibers of the NIRS apparatus 416. The detectors 432 (e.g., spectrometers 346, 348, 350) are optically coupled to a remaining portion of the optical fibers of the NIRS apparatus 416. The NIRS control apparatus 426 is configured to perform NIRS (e.g., to illuminate and acquire spectroscopic information for the tissue) and provide corresponding NIRS data to the computer system 406 representative of one or more optical properties of the tissue.

In addition to the NIRS data and the OCT data, the computer system 406 can also receive sensor data from the one or more sensors 420 through the sensor interface(s) 412. The one or more sensors 420 can include temperature sensors, acoustic sensors, force or contact sensors, electrodes (e.g., for sensing electrophysiology signals, sensing impedance, and/or position sensing) and/or other sensors useful for controlling ablation or identifying one or more pathological regions for treatment. The computer system 406 can also receive operating parameter data from the controller 408, such as one or more parameters measured or otherwise determined during delivery of ablation energy (e.g., voltage, current, pulse width, frequency). The types of parameter data can vary depending on the type of ablation energy that the controller and generator are configured to deliver to the ablation electrode(s) 418.

The computing system 406 can include a processor 440 and non-transitory memory 442 to store instructions and data. The instructions are executable by the processor to perform the functions and methods described herein. The data stored in the memory 442 can include received data (e.g., the OCT data, such as OCT image data or other OCT measurements, the NIRS data, the sensor data, and the parameter data) as well as data computed based on the received data. The computer system 406 can evaluate the received data individually and/or in any combination thereof and provide one or more outputs. The computer system 406 can provide the one or more outputs to the controller 408, which can include or be derived from the NIRS data, the OCT data, the sensor data, and/or parameter data. Also, or alternatively, computer system 406 can provide the one or more outputs to an output device, such as representing a graphical output visualization for rendering on a display device, which can include or are derived from the NIRS data, the OCT data, the sensor data, and/or parameter data.

The controller can include (or be coupled to) a generator 436, which can be electrically coupled to the ablation electrode 418. The generator is configured to deliver ablation energy to the ablation electrode based on the ablation control signal. The generator 436 is configured to deliver radiofrequency (RF) energy to the tissue. Also, or alternatively, the generator 436 is configured to deliver pulse field ablation (PFA) to the tissue, including to perform reversible and/or irreversible electroporation. In an example, the generator 436 can be configured to modulate delivery of pulses of monophasic or biphasic energy based on optical feedback from tissue, such as described herein (e.g., based on OCT data and/or NIRS data). In other examples, the generator can also be configured to deliver other types ablation energy to the electrode or other corresponding features that can be implemented at the catheter 402, such as thermal energy, ultrasound energy, electromagnetic radiation (e.g., optical energy, such as laser ablation).

As an example, the instructions can cause the processor to compute at least one property of the sample and/or the ablation catheter based on at least one of the OCT image data and the NIRS data. The computed property can include one or more optical properties of the sample including, for example, one or more of absorption, scattering, refractive index, emission (e.g. fluorescence), polarization characteristics (including birefringence, local optic axis, diattenuation, etc.) at particular wavelengths or as a function of wavelength (e.g., spectroscopy), and/or as a function of location (e.g., imaging). In examples, where the sample includes biological tissue (e.g., cardiac tissue, such as endocardium, myocardium), the computed property can include one or more tissue property of the biological tissue, derived from measured optical properties, such as tissue thickness, fiber orientation, tissue health, estimated fibrotic percentage, collagen content, blood perfusion, biomolecular content, a quality of treatment or overtreatment, and/or estimated adipose percentage.

Additionally, in some examples, the computing system 406 can be configured to provide control signals to the controller 408 for titrating the effective dose of the ablation energy based on one or more of the computed properties, such as tissue thickness. For example, one or more tissue properties can define input parameters for feedback control loops (e.g., controls executed by the controller 408 and/or the computer system 406) to control parameters for delivery of ablation energy to the ablation electrode 418. Also, or alternatively, the computer system 406 can generate an output visualization on a display based on the computed properties (e.g., tissue thickness), which a practitioner can use (e.g., manually—in an open loop manner) to titrate the ablation dose responsive to a user input.

As a further example, the computer system 406 can include instructions programmed to compute one or more other properties of a tissue sample, such as fiber orientation, tissue health, fibrosis, and collagen content, to identify one or more target areas of ablation. Also, or as an alternative, the computer system 406 can include instructions programmed to identify potential drivers of an arrhythmia (e.g., atrial fibrillation) based on the one or more properties. The computer system 406 further can include instructions programmed to mark the location (e.g., 3D spatial coordinates) for the driver and/or ablation target on a 3D mapping system, such that optical and/or tissue properties derived from the NIRS data and/or OCT data can be superimposed on a 3D electroanatomical map to provide further guidance. The sample properties can be used in system feedback loops, such as described herein. Additionally, logging the ablation targets and pairing them with outcomes (through remote monitoring or other means) can provide feedback to the user on how effective the treatment paradigm is at treating the target area, such as for treating atrial fibrillation or other arrhythmias.

In some examples, the received data (e.g., including the OCT data and the NIRS data) are acquired over at least one time interval, and the computing system 406 is configured to evaluate changes in the OCT data and/or NIRS data or a property of the sample derived from the received data. As used herein, the changes can correspond to an absolute change in the at least one property over the at least one time interval, a relative change in the at least one property over the at least one time interval, or a rate of change in the at least one property over the at least one time interval. For example, one or more optical and/or tissue properties, which are determined based on the OCT data and/or NIRS data acquired during delivery of ablation energy to target tissue, could change over the time interval and the change can be used to control ablation and/or irrigation through the catheter 402.

As a further example, the computer system 406 can be configured to compute one or more indices based on any combination of the OCT image data, NIRS data, sensor data and/or parameter data. The one or more indices can characterize properties or attributes of the tissue (e.g., before, during, and/or after ablation), properties or attributes of the catheter 402, and/or properties or attributes of interactions between the catheter and the tissue, such as described herein. Examples of some indices include a contact index representative of a quality of contact between the ablation catheter and the sample, a health index representative of a health of a tissue sample, a thickness index representative of a thickness of the sample, an index representative of lesion completeness, an index representative of lesion formation (e.g., the LOI), an index indicative of a quality of treatment, an index indicative of overtreatment, and an index indicating one or more spatial gaps in lesion formation. The computer system 208 can determine other indices based on the OCT image data, NIRS data, other sensor data and/or parameter data. The index (ices) can be used to enable a practitioner to implement manual or semiautomated control of ablation using a multi-modal ablation catheter. Also, or alternatively, the index (ices) can be used as feedback to implement closed loop (e.g., automated) control for ablation. For example, the computer system can provide data or instructions to the controller 408 for implementing control based on one or more such indices.

By way of example where the tissue sample includes biological tissue (e.g., cardiac tissue, such as endocardial tissue), the computer system 406 can include instructions to compute an index representative of tissue thickness and/or tissue health based on the OCT image data and the NIRS data. The computer system 406 can set a threshold for termination of ablation based on the computed index. The controller 408 can control delivery of ablation energy to the ablation electrode based on the threshold. For example, during application of ablation energy to a target region of the tissue, OCT image data and/or the NIRS data can be acquired for the target region and the computer system can compute a value based on the acquired OCT image data and the NIRS data. In response to detecting that the index satisfies the threshold, the computing system can command the controller to terminate application of ablation energy to the ablation electrode 418. Also, or alternatively, one or more of the other indices described herein likewise can be used to control delivery of ablation energy to the electrode for ablating tissue. In some examples, the instructions executed by the processor 440 for computing one or more indices can include a machine learning model trained to perform the computing and generate a respective index (ices) and/or corresponding thresholds based on the OCT image data, the NIRS data, sensor data, and/or parameter data.

As an example, the instructions executed by the processor 440 can include a model that has been trained to provide results data that include one or more properties (e.g., optical properties and/or tissue properties), such as described herein, based on OCT data, NIRS data, or a combination of OCT data and NIRS data. Also, or alternatively, a model can be trained to model that has been trained to determine one or more indices one or more properties (e.g., optical properties and/or tissue properties), such as described herein, based on OCT data, NIRS data, or a combination of OCT data and NIRS data. The model can be trained according to any of a variety of techniques for generating models, including support vector machines, regression models, self-organized maps, k-nearest neighbor classification or regression, fuzzy logic systems, data fusion processes, boosting and bagging methods, rule-based systems, artificial neural networks, and/or deep learning methods.

The computer system 406 further can be configured to control the OCT control apparatus 422 for performing OCT on tissue based on NIRS data acquired by the NIRS control apparatus for such tissue. Also, or alternatively, the computer system 406 can control the NIRS control apparatus for performing NIRS on tissue based on OCT data acquired by the OCT control apparatus for such tissue.

The computing system 406 can also be configured to provide irrigation data to control the fluid source 410 to deliver irrigation fluid to the ablation catheter (e.g., into volume 119) based on the OCT data, NIRS data, sensor data and/or based on data (e.g., one or more indices) derived from such data, such as described herein. For example, the fluid source 410 includes an irrigation controller and a pump configured to control delivery of an irrigation fluid into the ablation catheter based on the irrigation data.

As a further example, while the examples above have been described with respect to including NIRS and OCT (e.g., conventional OCT and/or PSOCT), the systems and methods described herein further can be implemented in combination with one or more other sensing modalities. For example, the systems and methods can be combined with electroanatomical mapping and/or medical imaging modalities (e.g., x-ray, ultrasound, etc.) to further help users to identify regions of potentially pathological tissue substrate by using low voltage thresholds to measure slowly conducting regions.

In view of the foregoing, the systems and methods here can leverage optical interactions with the tissue to inspect fibrosis, adipose, myocardium, and atrial wall structure, and identify tissue locations to target for ablation or to avoid ablating. As the tissue changes during an ablation, the multi-modal optical measurements can assess lesion completeness, diameter, depth, and prevent injury. As a lesion is formed, proteins denature and optical properties of the tissue change. PSOCT-NIRS can measure and image these characteristics to provide boundaries of where tissue is ablated and not-ablated. This intraoperative monitoring can allow the user to see a lesion form, monitor gaps or non-complete lesions, and prevent injury in multiple ways. For example, the OCT data and NIRS data can be evaluated to identify or predict a steam pop, which occurs when the tissue is heated above 100° C. PSOCT can image microbubbles being formed before a steam pop occurs or the tissue is perforated completely. Further, the combination of PSOCT-NIRS can assess atrial thickness. PSOCT is accurate in measuring the atrial thickness within its depth range of a few millimeters. However, there are regions of the heart where the wall is thicker than PSOCT's imaging depth. This can be supplemented by NIRS. Combining measurements (e.g. endocardial thickness, tissue attenuation) from PSOCT and spectral measurements from NIRS allows for thickness measurements in the entire atria (or other tissue region of interest). This will allow clinicians to better plan ablation treatment by knowing where thin tissue and thick tissue are located across a target tissue region. The systems and methods herein thus can identify thin regions of the heart and mitigate risk when ablating such regions.

Additionally, existing approaches for selecting adjunctive ablation sites, electroanatomical mapping, is sensitive to inconsistent contact force, assumes tissue thickness, and does not measure fiber orientation. Further, electroanatomical mapping measures myocardium activity and not substrate that causes pathology-fibrosis and adipose. PSOCT-NIRS can assess (e.g., continually) whether the tissue is in contact with the catheter, and utilizing the systems and methods described herein, PSOCT-NIRS is able to assess contact at many different appositions of the catheter to the atrial wall. Combining both PSOCT-NIRS modalities, enables accurate tissue thickness to be acquired and incorporated into algorithms for detecting pathology. Furthermore, fiber orientation is measurable using PSOCT to detect the optic axis of birefringent media like the myocardium. This provides a more informative dataset for the orientation and/or degree of organization of myocardium with respect to low voltages or conduction patterns. PSOCT-NIRS is also capable of defining tissue morphology and content. Directly quantifying fibrosis and adipose will be beneficial to understanding pathology and more accurately defining regions for targeted ablations.

Additionally, PSOCT-NIRS can also be a valuable tool in the septal crossing from right atrium to left atrium. In most curative procedures, catheters are introduced through the right atrium but need to treat regions in the left atrium. A transseptal crossing is usually assisted by fluoroscopy, intracardiac ultrasound, and pressure sensing to determine whether the catheter is in the right or left atrium. The fossa ovalis is the thinnest part of the septum between the atria and is the ideal crossing point. PSOCT imaging can identify the thinnest section of the wall with high resolution because the region is less than a millimeter thick. NIRS further can monitor blood oxygenation which is different in the right and left atrium. Combined, the catheter described herein can be used to identify optimal regions for septal crossing and indicate to the clinician when the crossing is complete.

In a procedure to ablate a region for treating atrial fibrillation, the clinician steers an ablation catheter into the left atrium and ablates one or more target tissue regions. Each ablated tissue region is a thermally damaged section of tissue that is meant to be non-conductive to cardiac signals. In this way, the clinician can create an isolating fence around regions of the heart that generate ectopic signals that would otherwise initiate an erroneous heartbeat. The multi-modal catheter described herein can be used to acquire OCT and NIRS data to provide complementary metrics with respect to ablation treatment, such as for measuring full atrial thickness, predicting relevant lesion gaps or incompleteness, and inform the clinician's choice of power and/or duration for an ablation. Advantageously, OCT and NIRS measurements can be made (e.g., using multi-modal catheter 100, 302, 402) simultaneously and at the same position as a lesion develops.

In view of the foregoing, devices, systems, and methods provide a catheter capable of making NIRS measurements, OCT (e.g., PSOCT) measurements, and ablation measurements. Such measurements can be acquired simultaneously for a single region of interest, which can include formation of a single controlled lesion. The devices, systems, and methods further can provide intraoperative feedback for ablation treatment of atrial fibrillation and other arrhythmias.

In view of the foregoing structural and functional description, those skilled in the art will appreciate that portions of the invention may be embodied as a method, data processing system, or computer program product. Accordingly, these portions of the invention may take the form of an entirely hardware embodiment, an entirely software embodiment, or an embodiment combining software and hardware. Furthermore, portions of the invention may be a computer program product on a computer-usable storage medium having computer readable program code on the medium. Any suitable computer-readable medium may be utilized including, but not limited to, static and dynamic storage devices, hard disks, optical storage devices, and magnetic storage devices.

Certain embodiments of the invention have also been described herein with reference to block illustrations of methods, systems, and computer program products. It will be understood that blocks of the illustrations, and combinations of blocks in the illustrations, can be implemented by computer-executable instructions. These computer-executable instructions may be provided to one or more processor of a general purpose computer, special purpose computer (e.g., as part of the imaging system), or other programmable data processing apparatus (or a combination of devices and circuits) to produce a machine, such that the instructions, which execute via the processor, implement the functions specified in the block or blocks.

These computer-executable instructions may also be stored in computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims.

As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

Spatially relative terms, such as “under,” “below,” “lower,” “over,” “upper”, “proximal”, “distal”, and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

All references, publications, and patents cited in the present application are herein incorporated by reference in their entirety.

What is claimed is:

1. An ablation catheter, comprising:
an elongate tubular body having a distal tip portion, defining an ablation electrode, and a shaft extending proximally from the distal tip portion to a proximal end of the shaft, in which a central lumen extends through the shaft and the distal tip portion to provide a central opening at a distal end of the distal tip portion, the ablation electrode has a substantially cylindrical body circumscribing the central lumen and extending axially from a distal end of the shaft to terminate at the distal end of the distal tip portion, and the ablation electrode includes an arrangement of apertures extending through the distal tip portion radially outwardly from the central opening;
an optical coherence tomography (OCT) imaging probe extending within the central lumen of the elongate tubular body and terminating in an optical assembly at a distal end thereof that is at or spaced proximally from the distal end of the distal tip portion; and
a near-infrared spectroscopy (NIRS) apparatus comprising a plurality of optical fibers, in which each of the optical fibers extends longitudinally through the elongate tubular body spaced from the OCT imaging probe and terminates in a respective distal end within at least one of the apertures in the distal tip portion.

2. The ablation catheter of claim 1, wherein the apertures are distributed substantially evenly and circumscribe an inner periphery of the central opening such that distal ends of the respective optical fibers are circumferentially around the optical assembly of the OCT imaging probe.

3. The ablation catheter of claim 1, wherein at least the optical assembly of the OCT imaging probe is configured to rotate about a central longitudinal axis extending through the central lumen, and the OCT imaging probe is configured to transmit light off-axis through the central opening in at least one of a substantially conical or cylindrical pattern.

4. The ablation catheter of claim 1, wherein the distal tip portion comprises:
a first portion having a substantially cylindrical first sidewall, in which the first sidewall has an inner periphery and an outer periphery, and the first portion has a distal end through which the apertures and the central opening extend; and
a second portion having a substantially cylindrical second sidewall, in which the first sidewall has an inner periphery and an outer periphery and extends between proximal and distal ends thereof, the central lumen extends axially through a central portion of the second sidewall and defining at least a portion of the inner periphery of the second sidewall, at least a portion of the outer periphery of the second sidewall is spaced radially inwardly from and coextensive with the inner periphery of the first sidewall to define an interior volume of the distal tip portion between the first portion and the second portion, and the inner periphery of the second sidewall is configured to receive therein a distal portion of the optical assembly, and the proximal end of the sidewall of the second portion is coupled to the distal end of the shaft.

5. The ablation catheter of claim 4, wherein the proximal end of the second portion comprises an annular base extending radially outwardly from the second sidewall, in which the annular base includes an arrangement of guide apertures extending through the annular base axially aligned with at least some of the respective apertures at the distal end of the first portion.

6. The ablation catheter of claim 4, further comprising one or more lumens extending longitudinally through the shaft and in fluid communication with the volume, in which one or more holes extend through the first sidewall to enable flow of fluid between the volume and outside of the distal tip portion of the catheter.

7. The ablation catheter of claim 4, wherein the first portion and the second portion are formed of electrically conductive materials and are electrically coupled to each other.

8. The ablation catheter of claim 4, wherein the first portion and the second portion are electrically isolated from each other.

9. A system comprising the ablation catheter of claim 1, the system comprising:
an OCT control apparatus optically coupled to or including the OCT probe, the OCT control apparatus configured to perform polarization sensitive OCT imaging and provide OCT image data representative of one or more optical properties of a sample within a field of view of the OCT probe,
wherein the NIRS apparatus comprises light source and a detector, in which the light source is optically coupled to at least one of the optical fibers, the detector is optically coupled to a remaining portion of the optical fibers, and the NIRS apparatus is configured to perform NIRS and provide NIRS data representative of one or more optical properties of the sample.

10. The system of claim 9, further comprising:
a controller configured to provide an ablation control signal based on at least one of the OCT image data and the NIRS data; and
a generator electrically coupled to the ablation electrode, the generator configured to deliver ablation energy to the ablation electrode based on the ablation control signal. The ablation energy can include radiofrequency (RF) or pulse field ablation (PFA), including to perform reversible and/or irreversible electroporation.

11. The system of claim 9, further comprising:
a processor; and
non-transitory memory to store instructions and data, the data including the OCT image data and the NIRS data, the instructions being executable by the processor to perform a method, the method comprising:
computing at least one property of the sample and/or the ablation catheter based on at least one of the OCT image data and the NIRS data.

12. The system of claim 11, wherein the at least one computed property includes at least one optical property of the sample comprising one or more of birefringence, local optic axis, absorption, scattering, spectral shape, and reflectance spectrum.

13. The system of claim 11, wherein the sample includes biological tissue and the at least one computed property includes at least one tissue property of the biological tissue comprising at least one of tissue thickness, fiber orientation, tissue health, estimated fibrotic percentage, collagen content, a quality of treatment or overtreatment, or estimated adipose percentage.

14. The system of claim 11, wherein the OCT image data and the NIRS data are acquired over at least one time interval, and at least one computed property of the sample are determined based on at least one of an absolute change in the at least one property over the at least one time interval, a relative change in the at least one property over the at least one time interval, or a rate of change in the at least one property over the at least one time interval.

15. The system of claim 11, wherein the method further comprises computing an index characterizing the at least one property of the sample and/or the ablation catheter.

16. The system of claim 15, wherein the index is one of a contact index representative of a quality of contact between the ablation catheter and the sample, a health index representative of a health of a tissue sample, a thickness index representative of a thickness of the sample, an index representative of lesion completeness, an index indicative of a quality of treatment or overtreatment, and an index indicating one or more gaps in lesion formation.

17. The system of claim 11, wherein the sample includes biological tissue, and the method further comprises:
computing tissue thickness and/or tissue health based on the OCT image data and the NIRS data;
setting a threshold for termination of ablation based on the tissue thickness and/or tissue health; and
control delivery of ablation energy to the ablation electrode based on the threshold.

18. The system of claim 11, wherein the instructions include a machine learning model trained to perform the computing based on at least one of the OCT image data and the NIRS data.

19. The system of claim 9, further comprising:
an irrigation controller configured to control delivery of an irrigation fluid into the ablation catheter based on at least one the OCT image data and the NIRS data.

20. A method comprising:
positioning a distal tip portion of a catheter proximal a region of interest of biological tissue, in which the catheter includes an elongate tubular body that terminates at the distal tip portion, at least a portion of the distal tip portion defines an ablation electrode of the catheter, and the distal tip portion includes:
an optical coherence tomography (OCT) imaging probe extending through a central lumen of the tubular body and terminating in an optical assembly at a distal end thereof that is at or spaced proximally from the distal end of the distal tip portion; and
a near-infrared spectroscopy (NIRS) apparatus comprising a plurality of optical fibers extending longitudinally through the elongate tubular body spaced radially outwardly from the OCT imaging probe and terminating in a respective distal end thereof within the distal tip portion;
performing OCT imaging with the OCT imaging probe to provide OCT image data representative of one or more optical properties of the biological tissue within a field of view of the OCT probe;
performing NIRS with the NIRS apparatus to provide NIRS data representative of one or more optical properties of the biological tissue within a field of view of the NIRS apparatus; and
controlling delivery of ablation energy to the ablation electrode based on at least one of the OCT image data and the NIRS data.

21. The method of claim 20, further comprising computing at least one property of the biological tissue and/or the catheter based on at least one of the OCT image data and the NIRS data, wherein controlling delivery of ablation energy to the ablation electrode is based on the at least one property of the biological tissue.
